(19) United States
(12) Patent Application Publication
(10) Pub. No.: US 2023/0172132 A1
(43) Pub. Date: Jun. 8, 2023

(54) A METHOD FOR INCREASING BETALAIN CONTENT IN A CROP PLANT

(71) Applicant: SENSIENT COLORS, LLC, St. Louis, MO (US)

(72) Inventors: Vergel C. CONCIBIDO, St. Louis, MO (US); Joerg MEYER, St. Louis, MO (US); Bradley LAVALLEE, St. Louis, MO (US); Timothy Mark IVANCIC, St. Louis, MO (US); Jessica Kathleen LOBO, St. Louis, MO (US); Michael Charles GERAGHTY, St. Louis, MO (US); David Eldridge GEBHARDT, St. Louis, MO (US); Alejandra VALDEZ ZARCO, St. Louis, MO (US); Gian Carlo LEOCATA, St. Louis, MO (US)

(21) Appl. No.: 17/923,369

(22) PCT Filed: May 4, 2021
(86) PCT No.: PCT/US2021/030633

§ 371 Date: Nov. 4, 2022

Related U.S. Application Data
(60) Provisional application No. 63/019,805, filed on May 4, 2020.

(51) Int. Cl. A01H 3/04; A01N 43/40; A01P 21/00; C09B 61/00; A23L 5/43

(52) U.S. Cl. CPC A01H 3/04; A01N 43/40; A01P 21/00; C09B 61/00; A23L 5/43

(57) ABSTRACT

The methods described herein relate to the isolation of betalam pigment color compositions from crop plants. Also described are methods of pre-harvest foliar spraying of an ethylene-releasing compound to a crop plant and the uses of extracted betalam pigment color compositions from crop plant for a food or commercial product.

### FIELD OF THE INVENTION

The present invention relates to a method for obtaining a betalain pigment color composition from crop plants comprising a method of pre-harvest foliar spraying of an ethylene-releasing compound to a crop plant and the uses of extracted betalain pigment composition from a crop plant for a food or commercial product.

### BACKGROUND OF THE INVENTION

Legal restrictions and consumer consumer concerns has led to an increased demand for natural food colorants which can be substituted for synthetic colors. For example, black carrot (Daucus carota subsp. sativus) and cultivars thereof, e.g., Antonina, Beta Sweet, Deep Purple, and Purple Haze. are a valuable source of carbohydrates, minerals and vitamins and contains also high amounts of anthocyanins giving the characteristic deep-purple color. (Montilla et al. J Agric Food Chem. 2011 Apr. 13; 59(7):3385-90).

Betalains are water-soluble, nitrogenous pigments which are indole derived phytochemicals. Betalains can be subdivided into two structural groups; betacyanins (red-blue pigments) and betaxanthins (yellow-orange pigments). Currently, the extracts of various cultivated crops are widely utilized in juices, confectionery, candies, ice cream, and soft drinks. The betalains are well-known as a group of compounds which give color to food, vegetables, and flowers, and are responsible for the magenta, red, orange, and yellow color of many plants in the order of Caryophyllales as well as in several species of fungi (Rahini et al. Crit Rev Food Sci Nutr, 59 (18) 2019, 2949-2978). In beet crops, for example, the principle betacyanins are betanin and isobetanin, and the principle betaxanthin is vulgaxanthin. However, there are other betacyanins and betaxanthins which exist in beet crops in smaller proportions.

The betalains are characterized as non-toxic pigments, and therefore the betacyanins and betaxanthins extracted from fruits and vegetables have been used as food colorants to provide color in the yellow to red-blue color range.

Betalains are regarded as secondary metabolites and antioxidants. The accumulation of secondary metabolites in a crop plant requires elicitors, which usually act as signaling molecules in stress responses of a crop plant. There are certain compounds in the art promote the release of ethylene when sprayed on crop plants. These compounds are called ethylene-releasing compounds (ERC) and accelerate several physiological responses in a crop plant such as induction of flowering, stimulation of latex flow, leaf and branchlet abscission, fruit ripening, fruit abscission, and pod dehiscence. Examples of such ethylene-releasing compounds are known in the art and include, but are not limited to ACC, Ethephon, Glyoxime, Etacelasil, Silaid, and Alsol, ACC (M. S. Reid, Plant Growth Substances 1988 pp 595-603). Ethephon, for example, has been used to improving carotene contents in orange carrots and to accelerate the advancement of maturity and anthocyanin contents during apple ripening.

### SUMMARY OF THE INVENTION

The disclosure provides a method for increasing betalain pigment color composition from a crop plant, notably a beet plant of the varieties Ruby Lake, Red Cloud, Ruby Queen, Nochowski, Chioggia, and Monti F1 wherein the method increases the yield and or amount of betalain pigment.

In one embodiment, the method comprises the pre-harvest foliar spraying of an ethylene-releasing compound on a beet plant, wherein the pre-harvest foliar spraying of an ethylene-releasing compound leaves the mean betalain concentration in the roots of the treated plants increased by more than 200% when compared to controls without any spraying.

Accordingly, a first aspect of the invention relates to a method for obtaining a betanin and vulgaxanthin pigment color composition from a beet plant comprising the following steps:
(i): foliar spraying of an ethylene-releasing compound on the leaves of a beet plant
(ii): harvesting the beet plants of step (i); and
(iii): isolating one or more betalains from the harvested beet plants of step (ii), wherein the betalain is selected from betacyanin and betaxanthin pigment color compositions.

A second aspect of the invention relates to the use of a betalain pigment color composition obtained according to the method of the first aspect and/or an embodiment thereof for coloring of an edible food product.

### DESCRIPTION OF THE DRAWINGS

FIG. 1. Data shows greenhouse-applied ethylene-releasing compound induced changes in the accumulation of betacyanins (quantified in betanin equivalents) measured at time of harvest.

FIG. 2. Data shows greenhouse-applied ethylene-releasing compound induced changes in the accumulation of betaxanthins (quantified in vulgaxanthin equivalents) measured at time of harvest.

FIG. 3. Betanin/vulgaxanthin ratio from a greenhouse-applied ethylene-releasing compound.

FIG. 4. Colorant change of betanin, dry basis (mg/kg) in control versus Ethephon-treated beets.

FIG. 5. Colorant change of vulgaxanthin, dry basis (mg/kg) in control versus Ethephon-treated beets.

FIG. 6. Ry values for betalains in control versus Ethephon-treated beets.

FIG. 7. % Solids of root in control versus Ethephon-treated beets.

FIG. 8. Mass of root in in control versus Ethephon-treated beets.

FIG. 9. Betanin per root [% w/w] in control versus Ethephon-treated beets.

FIG. 10. Photographs of red beet, black carrots—cv. “Deep Purple” and red radish —“Chinese Variety” after spray administration of Ethephon at low and high dosages.

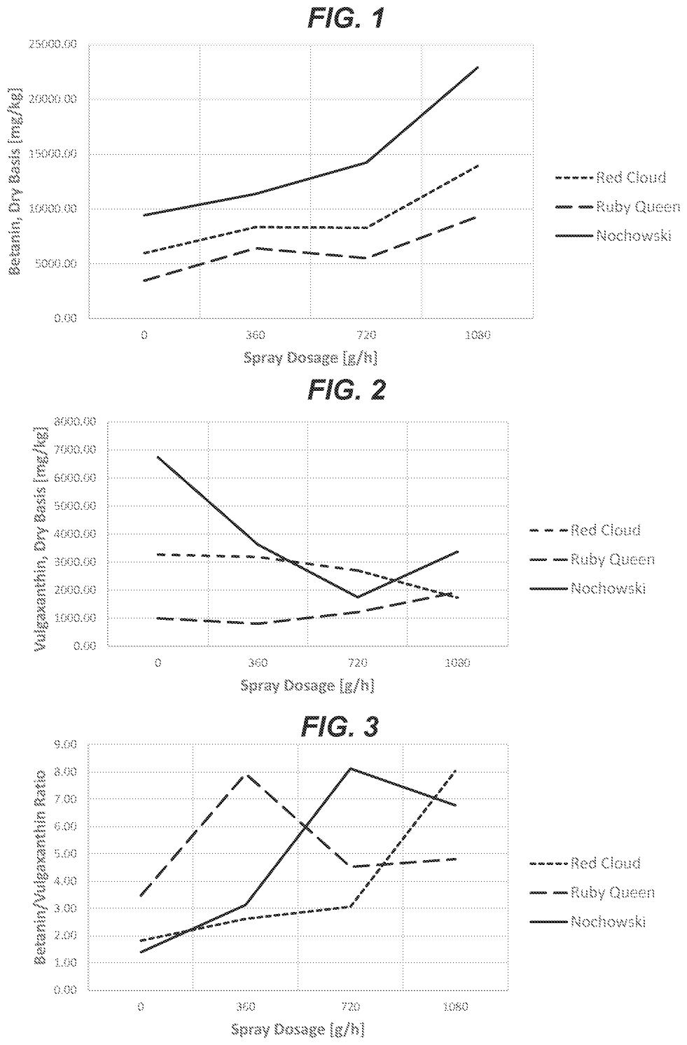

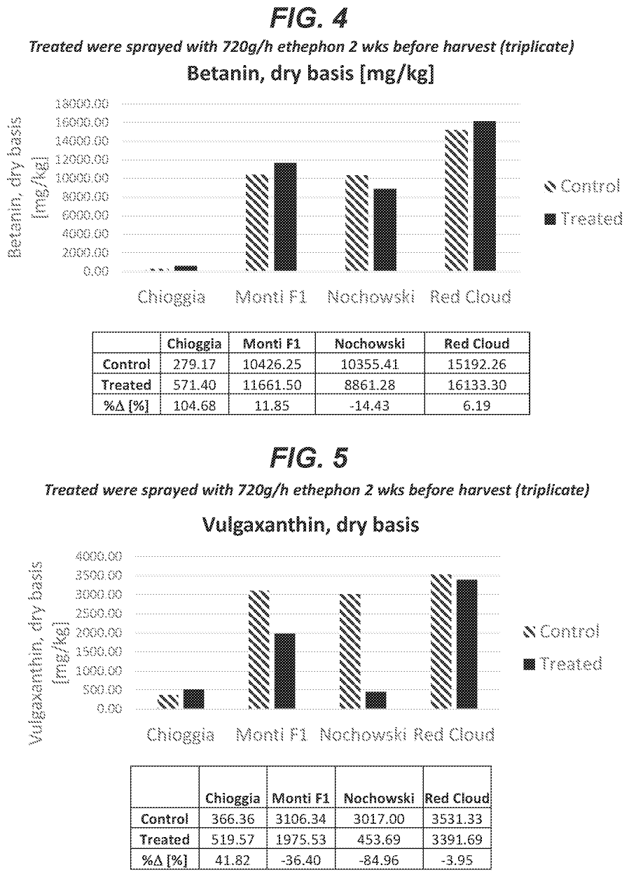

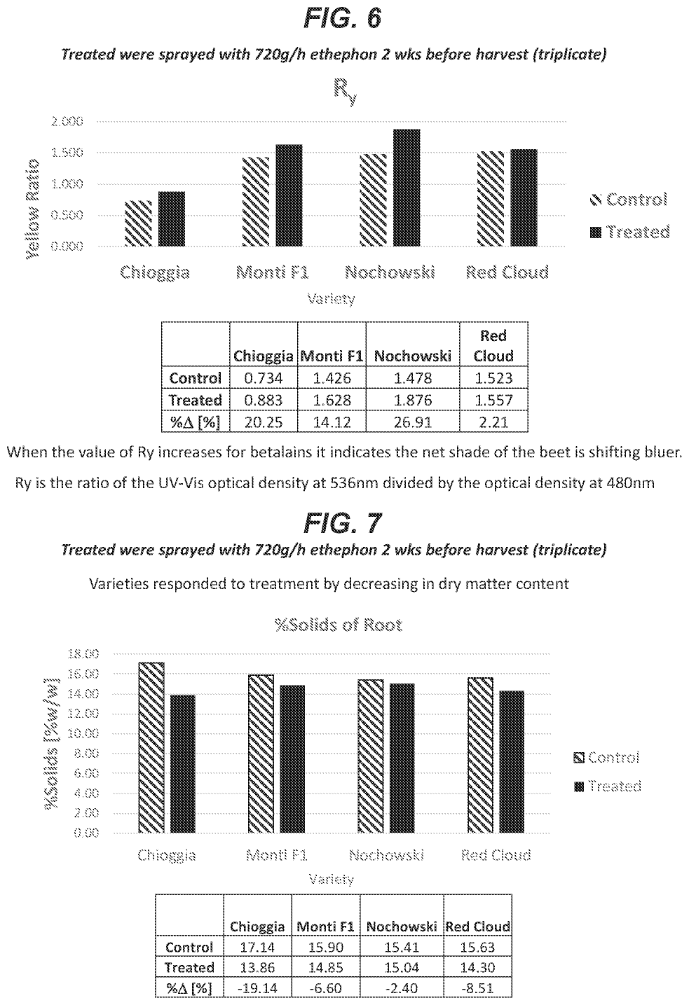

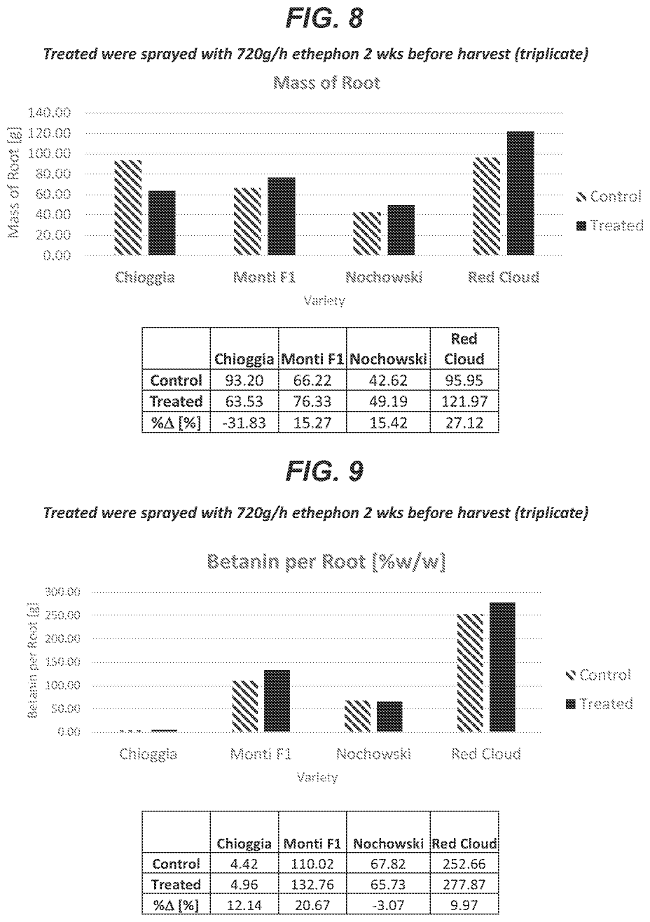

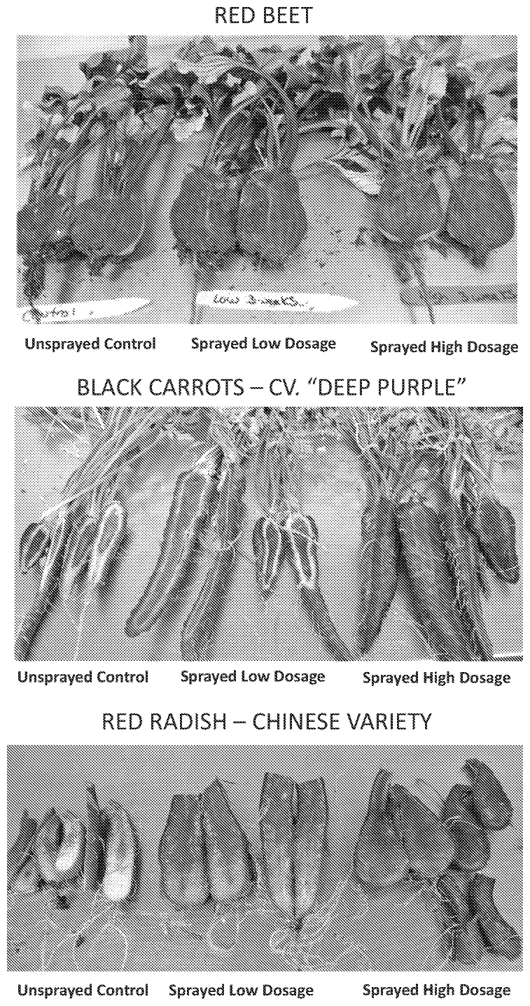

### DETAILED DESCRIPTION OF THE INVENTION

A method for obtaining a betalain pigment color composition from a beet plant:

As discussed above, a first aspect of the invention relates to a method for obtaining betalain pigment color composition from beet plants comprising the following steps:
(i): foliar spraying of an ethylene-releasing compound on the leaves of a beet plant;
(ii): harvesting the beet plant of step (i); and
(iii): isolating one or more betalains from the harvested beet plants of step (ii), wherein the betalain is selected from betacyanin and betaxanthin pigment color compositions.

It is evident that the beet plant herein is a beet plant that is capable of producing betacyanins, quantified in betanin equivalents, (red-blue pigments) and betaxanthins, quantified in vulgaxanthin equivalents, (yellow-orange pigments).

The beet plants as described herein, are selected from, but not limited to, the beet varieties described in Table 1, and are used in this invention.

TABLE 1
List of Exemplary Beet Varieties

| Beet Variety | Color |
|---|---|
| Agyptische Plattrunde | red |
| Akela Rz | red |
| Babybeet | red |
| Betina | red |
| Bohardy | red |
| Bolder | red |
| Bolivar | red |
| Bordo AS | red |
| Boro F1 | red |
| BoRu1 | red |
| Bulls Blood | red |
| Carillon Rz | red |
| Cervena Kulata | red |
| Ceryl | red |
| Chard | red |
| Chrobry | red |
| Cylindra | red |
| Czerwona Kula 2 | red |
| Detroit 2 Dark Red | red |
| Detroit 3 | red |
| Detroit Globe | red |
| Eagle | red |
| Early Blood Turnip Root | red |
| Early Wonder | red |
| Formanova | red |
| Forono | red |
| Gesche SG | red |
| Jannis | red |
| Kestrel F1 | red |
| Libero | red |
| Long season Pink Stein | red |
| Lutz Green Leaf | red |
| Merlin | red |
| Moneta (Monogerm) | red |
| Monty RZ F1 | red |
| Nobol | red |
| Nochowski | red |
| Pablo F1 | red |
| Red Ace | red |
| Red Bull | red |
| Red Cloud | red |
| Red Mommouth | red |
| Red Titan | red |
| Rhoda | red |
| Robuschka | red |
| Ronjana | red |
| Rote Kugel 2 Hilmar | red |
| Ruby Lake | red |
| Ruby Queen | red |
| Subeto | red |
| Sweet Dakota | red |
| UB-E3 | red |
| Zeppo | red |
| Chioggia | red- |
| | white |
| | stripes |
| Golden Beet | yellow |
| Golden Detroit | yellow |
| Golden Grex | yellow |
| Touchstone | yellow |

The term “foliar spraying” relates to a technique of feeding plants by applying liquid active ingredient (herein, ethylene-releasing compound) directly to their leaves.

Examples of ethylene-releasing compounds are known in the art and include, but are not limited to ACC, Ethephon, Glyoxime, Etacelasil, Silaid, and Alsol, ACC (M S. Reid, Plant Growth Substances 1988 pp 595-603). Other chemical compounds that can induce a result of increasing pigment production in plants are called elicitors. Elicitors can be natural or synthetic compounds. Elicitors can induce a physical change, e.g., the application of heat and cold, withdrawal of water, slight wounding, insect feeding, or inoculation with fungal, microbial or viral plant diseases. These additional elicitor compounds are described in Table 1.

TABLE 2
Elicitor compounds

| Ethylene-releasing | Examples of commercial | Product label claim on pigment |
|---|---|---|
| compound | agricultural products | enhancement. |
| Abscisic acid | ProTone SL | pigment enhancement in grapes |
| Auxins | PoMaxa | None |
| Auxins | Apogee | improved color of red apple |
| | | varieties because of better light |
| | | penetration into the canopy |
| Auxins | BioRoot | None |
| Auxins | Atrimmec | None |
| Benzoic acid | Lab products only | None |
| Benzo thiadiazole | CGA 245704 | None |
| Brassinosteroids | 14-hydroxylated brassinosteroid | None |
| Chitosan | Lab products only | None |
| Cytokinins | Miller Cytokin, Prestige | Cytokin is involved in all facets of |
| | | plant physiological production |
| | | periods from seed germination |
| | | through ripening on most crops. |
| Ethephon | Ethephon SL, Ethrel, Arvest, | Used in the acceleration of ripening |
| | Bromeflor, Etheverse, Flordimex, | of fruits and vegetables |
| | Flordimex T-Extra, Cerone, | |
| | Etherel, Chipco Florel Pro and Prep | |
| Gibberellin | Activol, Berelex, ProGibb, | None |
| | ProMalin, Regulex, Release, | |
| | RyzUp | |
| Jasmonates | Lab products only | None |
| Jasmonic acid | Lab products only | None |
| Methyl salicylate | Lab products only | None |
| Salicylic acid | Lab products only | None |
| Yeast extract | Danstar, Fleischmann's | None |

An “Ethylene-releasing compound” relates herein to a compound that release ethylene when sprayed on plants. The ethylene releasing compound is also denoted the “active ingredient”.

The chemical names for Ethephon, Silaid, Alsol, ACC are: Ethephon: 2-Chloroethylphosphonic acid; Silaid: (2-chloroethyl)methylbis(phenylmethoxy)silane; Alsol: (2-chloroethyl)tris(2-methoxyethoxy)silane; and ACC: 1-aminocyclopropane-1-carboxylic acid

In one embodiment the ethylene-releasing compound is 2-Chloroethylphosphonic acid; (2-chloroethyl)methylbis(phenylmethoxy)silane; (2-chloroethyl)tris(2-methoxyethoxy)silane or 1-aminocyclopropane-1-carboxylic acid. It may be relevant that two or more different ethylene-releasing compounds are used in combination, e.g. a mixture of Ethephon and Alsol for spraying on a crop plant, e.g., a beet plant.

In another embodiment, the ethylene-releasing compound is 2-Chloroethylphosphonic acid. As discussed above, Ethephon is a commercial product comprising the compound with IUPAC name: 2-Chloroethylphosphonic acid. Other names include, e.g., Bromeflor, Arvest or Ethrel.

In the Examples described herein, Ethephon is used as 2-Chloroethylphosphonic acid composition.

TABLE 3
List of Additional Botanical Targets for Ethylene-releasing Compounds and
Elicitor Applications.

| | | | Plant | |
|---|---|---|---|---|
| Crop | Scientific Name | Color | parts | Pigment class |
| Acai | Euterpe oleracea | purple, green | fruits | Anthocyanin |
| Alkanet | Alkanna tinctoria. | red, purple | roots | Alkannin |
| Annatto | Bixa orellana | red | seeds | Carotenoid |
| Avocado | Persia gratissima | green, yellow | fruits | Carotenoid |
| Black currant | Ribes nigrum | purple | fruits | Anthocyanin |
| Black rice | Oryza sativa | purple | seeds | Anthocyanin |
| Black sorghum | Sorghum bicolor | purple | seeds | Anthocyanin |
| Blackberry | Rubus fruticosus | blue | fruits | Anthocyanin |
| Bineberry | Vaccinium spp. | blue | fruits | Anthocyanin |
| Blue tansy | Tanacetum annuum | blue | petals | Anthocyanin |
| Buriti | Mauritia Jlexuousa | red, orange | fruits | Carotenoid |
| Butterfly pea | CH tori a lernatea | blue | petals | Anthocyanin |
| Cacao | Theobroma cacao | brown | husk | Anthocyanin |
| Calendula/Mari | Calendual officinalis | orange | petals | Carotenoid |
| gold | | | | |
| Carrot | Daucus carota | red | roots | Carotenoid |
| Carrot | Daucus carota | purple | roots | Anthocyanin |
| Carrot | Daucus carota | yellow’ | roots | Carotenoid |
| Carrot | Daucus carota | orange | roots | Carotenoid |
| Cashew apple | Anacardium | yellow, | fruits | Carotenoid |
| | occidentale | orange | | |
| Chamomile | Matricaria | yellow | petals | Carotenoid |
| | chamomilla | | | |
| Cherry | Prunus avium | red | fruits | Anthocyanin |
| Cora | Zea maydis | red, purple | ears | Anthocyanin |
| Cranberry | Vaccinium | red | fruits | Anthocyanin |
| | macrocarpon | | | |
| Dyer’s rocket | Reseda luteola | yellow | leaves, | Flavone |
| | | | seeds | |
| Elderberry | Samhucus nigra | red | fruits | Anthocyanin |
| Gardenia | Gardenia jasminoides | yellow, blue | fruits | Geniposide |
| Grapes | Vitis vinifera | red, purple | fruits | Anthocyanin |
| Green tea | Camellia sinensis | flavor | leaves | n/a |
| Hemp | Cannabis saliva | green | leaves | Chlorophyll |
| Henna | Lawsonia inermis | red, orange | leaves | Quinones |
| Indigo | Indigofera. tinctoria | blue, mauve | leaves | indican based |
| Iris | Iris germanica | purple, blue, | roots | Anthocyanin |
| | | green | | |
| Juito | Genipa americana | blue | fruits | Geniposide |
| Madder | Rubia tinctoriim | red, purple | roots, | Anthraquinone |
| | | | tubers | |
| Maqui berry | Aristotelia chilensis | purple | fruits | Anthocyanin |
| Monascus | Moua sen s purpureas | red, purple | fungi | Fungus |
| Nettie | Urtica dioica | green | leaf | Chlorophyll |
| Pansies | Viola tricolor var. | Multi-colored | flowers | anthocyaninand |
| | hortensis | | | carotenoid |
| Paprika | Capsicum ann uum | red, purple | fruits | Carotenoid |
| Pitaya/Dragon | Hylocerius undatus | purple | fruits | Betalain |
| fruit | | | | |
| Pomegranate | Punic a granatum | red, purple | seeds | Anthocyanin |
| Prickly pear | Opuntia elatior | Red, yellow | fruits | Betalain |
| Purple tomato | Lycopersicon | purple | fruits | Anthocyanin |
| | esculentum | | | |
| Purple yam | Dioscorea alata | purple | roots | Anthocyanin |
| Red beet | Beta vulgaris | red | roots | Betalain |
| Red cabbage | Brassica oleracea | pink, purple | heads | Anthocyanin |
| Red clover | Trifolium pratense | yellow | petals | Formonetin |
| Red radish | Raphan us sativum | red | roots | Anthocyanin |
| Red sandalwood | Pterocarpus | red | wood | Anthraquinone |
| | santalinus | | | |
| Red spicewood | Lindera benzoin | flavor | bark | n/a |
| Red tomato | Lycopersicon | red | fruits | Anthocyanin |
| | esculentum | | | |
| Rosehip | Rosa canina | red, orange | hips | Carotenoid |
| Roselle | Hibiscus sabdariffa | red, pink | calyces | Anthocyanin |
| Safflower | Cartham us tin ctorius | yellow, red | petals | Carthamins |
| Saffron | Crocus sativus | yellow, red | stigmas | Carotenoid |
| Sea buckthorn | Hippophae | orange | fruits | Carotenoid |
| | rhamnoides | | | |
| Spinach | Spinacia oleracea | green, yellow | leaves | Chlorophyll |
| Sweet potato | Ipomea batatas | red, purple | roots | Anthocyanin |
| Strawberry | Fragaria ananassa | red | | |
| Turmeric | Curcuma longa | yellow | rhizomes | Curcaminoid |
| Watermelon | Citrullus lanatus | red | fruits | Carotenoid |
| Yellow beet | Beta vulgaris | yellow | roots | Betalain |
| Wollberry/Goji | Lycium barbarum | red | fruits | Carotenoid |
| berry | | | | |

The term “harvesting” relates to the process of gathering a ripe crop from the fields.

The method described herein may be used for commercially-relevant, large scale production of a betalain pigment, e.g., the large scale isolation from a beet crop plant. Accordingly, it may be that the isolation of betalains of step (iii) is done from at least 15 different harvested beet plants, more preferably from at least 100 different harvested beet plants, even more preferably from at least 500 different harvested beet plants, or such as from at least 1000 different harvested beet plants. It is evident that when at least 15 different harvested beet plants are used in isolation step (iii), then foliar spraying of ethylene-releasing compound (e.g., 2-Chloroethylphosphonic acid) of step (i) has been done on at least 15 different beet plants and in step (ii), at least 15 different crop plants have been harvested. As discussed above, the present inventors identified that by pre-harvest foliar spraying of 2-Chloroethylphosphonic acid (e.g., 2-Chloroethylphosphonic acid) on beet leaves the mean betalain concentration of treated plants increased by an increased amount when compared to controls without any spraying.

In some embodiments, the method can be optimized, e.g., varying the amount of 2-Chloroethylphosphonic acid used in step (i), to get a maximum improvement of the yield/amount of isolated betalain pigment.

Preferably, the amount of isolated betalain pigment in step (iii) of the method is an amount of betalain pigment which is at least 15% higher (w/w), more preferably at least 20/a higher (w/w), even more preferably at least 25% higher (w/w), and most preferably at least 35% higher (w/w), as compared to the amount of betalain pigment that is isolated in a control experiment without use of the ethylene-releasing compound in step (i). The purpose of a control experiment is to analyze the effect of using ethylene-releasing compound (e.g. 2-Chloroethylphosphonic acid). Accordingly, everything in the control experiment, e.g., harvesting time in step (ii), the specific method of isolation in step (iii), is identical to the method of using ethylene-releasing compound, e.g., 2-Chloroethylphosphonic acid in the first aspect of the method.

In some embodiments, 2-Chloroethylphosphonic acid can be applied 6 weeks after planting/sowing, and continued every 3 weeks, for a total of 6 applications.

It is evident that an ethylene-releasing compound should not be applied by foliar spraying to the leaves before the leaves of beet plants have a relevant size.

Accordingly, and in relation to the method of step (i) it may be that the foliar spraying of an ethylene-releasing compound of step (i) is done later than 1 week after planting, preferably it is done later than 2 weeks after planting. It may be done later than 4 weeks after planting.

It may be preferred that the foliar spraying of ethylene-releasing compound of step (i) is done more than one time, e.g., 2 times before the harvesting the beet plants of step (ii). It may be that it is done at least 3 times before the harvesting the beet plants of step (ii), or at least 4 times, or at least 5 times.

Step (ii) of the first aspect of the method relates to harvesting the beet plants of step (i).

In some embodiments, harvesting the beet plants were done 7, 10, 13, 16, 19, 22, 25, 26, 29 and 35 weeks after planting and 2-Chloroethylphosphonic acid treated roots showed a higher mean betanin and mean vulgaxanthin concentration at every harvest point.

In relation to step (ii), it may be that harvesting of the crop plants of step (ii) of the first aspect of the method is done later than 4 weeks after planting, later than 6 weeks after planting, or it is done later than 10 weeks after planting.

Step (iii) of the first aspect of the method relates to isolating betalains from the harvested crop plants of step (ii) and thereby obtain the betalain pigment color composition. The term “isolating” in step (iii) should be understood as that some liquid (e.g. water) and/or solids are separated from the betalains, i.e., the betalain pigment color composition does not comprise all liquid (e.g. water) and/or solids of the carrots. For instance, in step (iii) obtained betalain pigment color composition may be a juice.

In some embodiments, the isolating step (iii), i.e., it may be done such as by extraction from the taproots of a population of harvested beet plants, for example, Ruby Lake, Red Cloud, Ruby Queen, Nochowski, Chioggia, and Monti F1.

It may, as in working Example described herein, that the isolation of the betalains of step (iii) is done by extraction of the betalain pigments from the taproots of the harvested crop plants, e.g., preferably, Ruby Lake, Red Cloud, Ruby Queen, Nochowski, Chioggia, and Monti F1).

In step (iii), the isolated betalain pigment color composition may be in liquid or dried form-Purification may be performed by High Performance Liquid Chromatography (HPLC) to obtain a desired degree of purity.

It may be preferred that the in step (iii) of the method the isolated betalain pigment color composition is a composition comprising at least 0.05% (w/w—dry matter) of the isolated betalain pigments, such as e.g. a composition comprising at least 0.5% (w/w—dry matter) of the isolated betalain pigments, or a composition comprising at least 2.5% (w/w—dry matter) of the isolated betalain pigments.

It may be preferred that the in step (iii) obtained betalain pigment color composition is a liquid composition or a dried composition that comprises less than 25% (w/w) of liquid (e.g., water).

The betalain pigment color composition for coloring of a product:

As discussed above, a second aspect of the invention relates to use of a betalain pigment color composition obtained according to the method of the first aspect and/or an embodiment thereof for coloring of an edible product or a pharmaceutical product.

Betacyanins and betaxanthins have been used as colorants for many products, e.g., food products, and the coloring use of the betalain pigment color composition of the second aspect may be performed. An edible product may be a food product or a feed product.

Examples of a food product are dairy product, juice, liquid beverage, powder beverage, confectionery, baked goods, processed foods, wine gum, marmalade, jam, sugar confectionery, panned chocolate lentils, sausage casings, pasta, macaroni, cheese, prepared food or extruded foods, and pet food.

The embodiments described herein can be further understood by reference to the following non-limiting examples.

### EXAMPLES

### Example 1

AnthocCanin Levels are Enhanced in Carrots, Beets, And Radishes with Ethehon Treatments

Planting of varieties were staggered across several weeks to vary maturity for one spray. Two spray rates were administered across all plants on one spray date (120 g/h and 360 g/h). Harvesting was done 3-4 weeks after spray. Initial visualization was performed prior to crop samples sent to laboratory for evaluation.

TABLE 4
Greenhouse Testing 1.

| | | | total | | | | Days from | |
|---|---|---|---|---|---|---|---|---|
| | | planting | plants | | | Spray | planting to | Harvest |
| Chamber 203 | plant date | week | planted | 120 g/h | 360 g/h | Date | spray | days |
| Black Carrot | 9-Oct | 1 | 16 | 8 | 8 | 24-Jan | 107 | 135 |
| Black Carrot | 15-Oct | 2 | 12 | 6 | 6 | 24-Jan | 101 | 129 |
| Purple Carrot | 9-Oct | 1 | 16 | 8 | 8 | 24-Jan | 107 | 135 |
| Purple Carrot | 15-Oct | 2 | 12 | 6 | 6 | 24-Jan | 101 | 129 |
| Red Beet | 9-Oct | 1 | 16 | 8 | 8 | 24-Jan | 107 | 135 |
| Red Beet | 15-Oct | 2 | 12 | 6 | 6 | 24-Jan | 101 | 129 |
| Red Beet | 23-Oct | 3 | 20 | 10 | 10 | 24-Jan | 93 | 121 |
| Red Beet | 29-Oct | 4 | 16 | 8 | 8 | 24-Jan | 87 | 115 |
| Red Radish | 7-Nov | 1 | 24 | 10 | 10 | 24-Jan | 78 | 106 |
| Red Radish | 27-Nov | 2 | 10 | 4 | 4 | 24-Jan | 58 | 86 |
| Yellow Beet | 9-Oct | 1 | 16 | 8 | 8 | 24-Jan | 107 | 135 |
| Yellow Beet | 15-Oct | 2 | 12 | 6 | 6 | 24-Jan | 101 | 129 |
| Yellow Beet | 23-Oct | 3 | 20 | 10 | 10 | 24-Jan | 93 | 121 |
| Yellow Beet | 29-Oct | 4 | 16 | 8 | 8 | 24-Jan | 87 | 115 |

Harvest date is 28 Feb. 2019;
Red Beet = Nochowski;
Yellow Beet = Touchstone

TABLE 5
Testing 1 Spray information.

Spray Information
1 Acre= 2.47 Hectares
Rates
120 g/h = 48.6 g/acre
360 g/h= 145.75 g/acre
120 g/h = 300 ppm Ethephon
360 g/h = 1000 ppm Ethephon
Spray tip (8002E)
30 PSI
calculated @ 10 gallons/acre
spray speed 4 MPH, 30 inches above
canopy

TABLE 6
Greenhouse Testing 2

| Lines | Planting | | Plants | | Plants | |
|---|---|---|---|---|---|---|
| Planted | date | Spray 1 | Sprayed/Rate | Spray 2 | Sprayed/Rate | Harvest |
| Nochowski | 18-Mar | 13-Jun | 6 | 27-Jun | 6 | 1-Aug |
| Red Cloud | 27-Mar | 13-Jun | 6 | 27-Jun | 6 | 1-Aug |
| Ruby | 1-Apr | 13-Jun | 6 | 27-Jun | 6 | 1-Aug |
| Queen | | | | | | |
| Chioggia | 18-Mar | 13-Jun | 6 | 27-Jun | 6 | 1-Aug |
| S&I carrots | 9-Mar | 13-Jun | 6 | 27-Jun | 6 | 1-Aug |
| Super | 18-Mar | 13-Jun | 6 | 27-Jun | 6 | 1-Aug |
| black | | | | | | |
| red radish | 9-May | 13-Jun | 3 | 27-Jun | 3 | 1-Aug |

Rates: 0 g/h, 360 g/h, 720 g/h, and 1080 g/h; 3 reps/2 plants per variety. 1 Acre = 2.47 Hectares.
Rates: 360 g/h = 145.75 g/acre; 720 g/h = 291.5 g/acre; 1080 g/h = 437.25 g/acre.
Rates: 360 g/h = 1000 ppm Ethephon, 720 g/h = 2000 ppm Ethephon, 1080 g/h = 3000 ppm

TABLE 7
Ethylene-releasing compound enhanced the
accumulation of betanins during red beet growth.

| | Betanin | Vulgaxanthin |
|---|---|---|
| | mg/kg, dry | [mg/kg, |
| Sample | basis] | dry basis] |
| GroceryStore | 5,000 to 9,000 | 1,000 to 2,000 |
| PurchasedRed | | |
| Beets | | |
| Nochowski | 9,400 | 6,700 |
| Ruby Queen | 3,500 | 800 |
| Red Cloud | 6,000 | 3,300 |
| Chioggia | 320 | 380 |

### Example 2

Adaptation Test of Five Commercial Varieties of Red Beet (Beta vulgaris L.) for the Production of Dye

The study was carried out in two locations in the south-central zone of the State of Mexico: San Bartolomé Atlatlahuca, Tenango del Valle and El Islote, Villa Guerrero. The town of San Bartolomé Atlatlahuca is geographically located at 19° 04 ′07′ ′North Latitude and 99° 36′ 40″ West longitude and at an altitude of 2680 masl; it presents a temperate sub-humid climate with rains in summer. El Islote is located at 18° 58 ′14″ North Latitude, 99° 39′ 38″ West Longitude and an altitude of 2217 masl; It has a temperate sub-humid climate with summer rains that start regularly in May and end in the first days of October.

Biological material. In the sowing of the two experiments, seeds of five beet varieties from Europe were used: Nochowski, Carillon, Forono, Ruby Queen, and Red Cloud (see Table 1). The Nochowski variety was used as a control.

TABLE 8
Description of the beet varieties evaluated in the study

| Variety | Root growth form | Germination (%) |
|---|---|---|
| 1. Nochowski | Round | 80-85 |
| 2. Carillon | Cylindrical | 90-95 |
| 3. Forono | Cylindrical | 90-95 |
| 4. Ruby Queen | Round | 90-95 |
| 5. Reed Cloud | Round | 90-95 |

Experimental design and treatments. The work was established under a randomized block experimental design with 5 treatments (five recently introduced commercial beet varieties), 4 replications and 20 experimental units 4 m long by 1.2 m wide (4.8 m²). The distribution of the treatments in the experimental plot is illustrated:

| | | | | | |
|---|---|---|---|---|---|
| Replication 1 | V3 | V2 | V5 | V4 | V1 |
| Replication 2 | V4 | V5 | V3 | V1 | V2 |
| Replication 3 | V2 | V3 | V5 | V4 | V1 |
| Replication 4 | V4 | V2 | V3 | V1 | V5 |

Establishment of the experiment. A week before the transplant, the land was cleaned, the soil was prepared and five seed beds were formed. Then the bottom fertilization was carried out and then the sowing was done. Four sowing lines were made on the back of the planting bed and the seeds were deposited every 10 cm at a depth of approximately 8 to 10 cm. The planting in the experimental plot of Tenango del Valle was made on Jul. 26, 2019, and in Villa Guerrero it was on Sep. 20, 2019. Subsequently, an application of insecticide and fungicide was carried out to prevent pests and diseases.

Fertilization. The nutrition of the beet culture was made with the formula 100N-80P-120K-60Ca-30Mg, which was composed with the following amounts of commercial fertilizers: 33 kg of Phosphonitrate, 174 kg of DAP 18-46-00, 200 kg of Potassium chloride, 228 kg of Tropicote (calcium nitrate) and 200 kg of Ultarsol Magnit (magnesium nitrate). The first application of fertilizer was made at planting and the second was applied one month after the first.

TABLE 9
Amounts of commercial fertilizer applied
in the first beet fertilization (42N-80P- 60K),

| Commercial | | | | |
|---|---|---|---|---|
| fertilizer | Quantity | Nitrogen | Phosphorus | Potassium |
| DAP 18-46-00 | 174 kg | 31.32 | 80.04 | 0 |
| Phosphonitrate | 33 kg | 10.89 | 0 | 0 |
| Potassium chloride | 100 kg | 0 | 0 | 60 |
| Total | 307 kg | 42.21 | 80.04 | 60 |

TABLE 10
Amounts of commercial fertilizer applied in the second
red beet fertilization (58N-00P-60K-50Ca-30 mg),

| Commercial | | | | | |
|---|---|---|---|---|---|
| fertilizer | Quantity | Nitrogen | Potassium | Calcium | Magnesium |
| Potassium chloride | 100 kg | 0 | 60 | 0 | 0 |
| Tropicote | 228 kg | 35.34 | 0 | 59.96 | 0 |
| Ultrasol Magnit | 200 kg | 23 | 0 | 0 | 30 |
| Total | 528 kg | 58.34 | 60 | 59.96 | 30 |

Conducting the experiments. In the experimental plot of Tenango del Valle, the weed was controlled manually with a hoe and the grass was controlled by the application of Fusilade (Fluazifop-p-butil) at a rate of 1.0 L/ha; while, in the Villa Guerrero plot it was only done manually. The prevention and control of pests and diseases of the root, stem and foliage in the established experimental plots, was carried out with periodic applications of chemicals recommended for horticultural crops.

TABLE 11
Chemicals applied for the prevention and control of red beet pests and diseases
in the experimental plot of Tenango del Valle.

| Commercial | | Dose/L | |
|---|---|---|---|
| product | Active ingredient | of water | Controlling pest or pathogen ntrola |
| Decis Forte | Deltametrina | 0.5 L | Defoliating worms (Spodoptera sp. and |
| (2)* | | | Trichoplusia ni), and whiteflies |
| | | | (Trialeurodes vaporariorum). |
| Prozycar (1) | Carbendazim | 1.0 g | Leaf spot (Alternaria sp. and Cercospora |
| | | | sp.). |
| Captan 50 (2) | Captan | 2.5 g | Mildew (Peronospora farinosa) and leaf |
| | | | spot (Alternaria sp. and Cercospora sp.), |
| Oxicob Mix | Oxiclorurode | 2.5 g | Mildew (Peronospora farinosa) and leaf |
| (3) | copper + Mancozeb | | spot (Alternaria sp. and Cercospora sp.). |
| Amistar 50 | Azoxystrobin | 0.5 g | Mildew (Peronospora farinosa), |
| (1) | | | powdery ash (Oidium spp.) Leaf spot |
| | | | (Alternaria sp. and Cercospora sp.). |

( )* Application number.

TABLE 12
Chemicals applied for the prevention and control of red beet pests and diseases
in the experimental plot of Villa Guerrero. Mexico.

| Commercial | | Dose/L | |
|---|---|---|---|
| product | Active ingredient | of water | Controlling pest or pathogen ntrolai |
| Decis Forte | Deltametrina | 0.5 L | Defoliating worms (Spodoptera sp. and |
| (2)* | | | Trichoplusia ni), and whiteflies |
| | | | (Trialeurodes vaporariorum). |
| Prozycar (1) | Carbendazim | 1.0 g | Leaf spot (Alternaria sp. and Cercospora |
| | | | sp.). |
| Captan 50 (2) | Captan | 2.5 g | Mildew (Peronospora farinosa) and leaf |
| | | | spot (Alternaria sp. and Cercospora sp.), |
| Oxicob Mix | Oxiclorurode | 2.5 g | Mildew' (Peronospora farinosa) and leaf |
| (3) | copper+Mancozeb | | spot (Alternaria sp. and Cercospora sp.). |
| Amistar 50 | Azoxystrobin | 0.5 g | Mildew (Peronospora farinosa), |
| (1) | | | powdery ash (Oidium spp.) Leaf spot |
| | | | (Alternaria sp. and Cercospora sp.). |

( )* Application number.

Betalain Content. In the experimental plot of Tenango del Valle, highly significant statistical differences (p≤0.01) were found between the red beet varieties in betacyanin content and significant for betalains (p≤0.05); There were no statistical differences (p≥0.05) between varieties in content of betaxanthins. The betalain content in beet roots is the result of the sum of betaxanthins plus betacyanins. In this plot, the four recently introduced red beet varieties and the control variety presented statistical equality in betaxanthin content, but were statistically different in betacyanin content, this resulted in the Reed Cloud and Ruby Queen varieties having the highest content of betalains than the control variety (Nochowsky) and the Forono and Carillon varieties, the latter produced the least amount of betalains. However, under the adverse environmental conditions of Tenango del Valle, the Reed Cloud variety produced the second best performance after the Nochowski control variety, while the Ruby Queen variety had the lowest yield.

TABLE 13
Average values of betaxanthins, betacyanins and betalains by variety
of red beet in the experimental plot of Tenango del Valle, Mexico.

| Treatment/variety | Betaxantins | Betacinain | Betalains (mg/L) |
|---|---|---|---|
| 1. Nochowski | 45.77 a | 102.34 a | 148.12 ab |
| 2. Carillon | 49.82 a | 73.39 b | 123.20 b |
| 3. Forono | 57.03 a | 92.62 a | 149.66 ab |
| 4. Ruby Queen | 60.32 a | 105.75 a | 166.07 a |
| 5. Reed Cloud | 66.61 a | 107.33 a | 173.94 a |

LSD Fisher (p ≤ 0.05) not significantly different.

In the Villa Guerrero experimental plot, the results obtained were very different from those of the Tenango del Valle plot, since there were significant statistical differences (P≤0.05) between the red beet varieties for content of betaxanthins, betacyanins and betalains. In this location, the Red Cloud variety outperformed the rest of the varieties tested in content of these three compounds, followed by the control Nochowski variety. Therefore, the Red Cloud variety presented the highest amount of betalains and Nochowsky variety came in second place; On the other hand, the Forono and Ruby Queen varieties had an intermediate content of betalains and presented statistical equality, the worst variety was Carillon (Table 14). Despite the fact that the Carillon variety showed the highest yield in this locality, its production of betalains was the lowest of all the beet varieties tested. In Villa Guerrero, the Red Cloud variety had a relatively high yield and had the highest production of betalains.

TABLE 14
Average values of betaxanthins, betacyanins, and betalains by
variety of red beet in the experimental plot of Villa Guerrero, Mexico.

| Treatment/variety | Betaxanthin | Betacyanins | Betalains (mg/L) |
|---|---|---|---|
| 1. Nochowski | 89.01 ab | 132.41 ab | 221.42 ab |
| 2. Carillon | 55.47 c | 85.98 c | 141.45 c |
| 3. Forono | 74.86 bc | 111.27 bc | 186.12 bc |
| 4. Ruby Queen | 61.98 c | 100.24 bc | 162.21 bc |
| 5. Reed Cloud | 100.82 a | 149.30 a | 250.12 a |

LSD Fisher (p ≤ 0.05) not significantly different.

When comparing the behavior of the five varieties in the two localities, it was observed that in Tenango del Valle, the Reed Cloud and Ruby Queen varieties outperformed the rest of the varieties in betalain content, but in Villa Guerrero the Reed Cloud variety was better than the remaining four varieties. However, in both locations the Reed Cloud variety showed greater stability and adaptation by producing higher root yield and higher betalain content. The Ruby Queen variety only produces a good yield under suitable or controlled environmental conditions, as in the town of Villa Guerrero.

TABLE 14
Average values of betalains by red beet variety and experimental plot.

| Treatment/variety | Tenango del Valle | Villa Guerrero |
|---|---|---|
| 1. Nochowski | 148.12 ab | 221.42 ab |
| 2. Carillon | 123.20 b | 141.45 c |
| 3. Forono | 149.66 ab | 186.12 bc |
| 4. Ruby Queen | 166.07 a | 162.21 bc |
| 5. Red Cloud | 173.94 a | 250.12 a |

LSD Fisher (p ≤ 0.05) not significantly different.

1. A method for isolating a betalain pigment color composition comprising:
a) foliar spraying an ethylene-releasing compound on the leaves of a beet plant;
b) harvesting the beet plant of step; and
c) isolating betalains from the harvested beet plants of step b, thereby obtaining the betalain pigment color composition.

2. The method of claim 1, wherein the beet plants are selected from the group consisting of Ruby Lake, Red Cloud, Ruby Queen, Nochowski, Chioggia, and Monti F1.

3. The method of claim 1, wherein the ethylene-releasing compound is selected from 2-Chloroethylphosphonic acid; (2-chloroethyl)methylbis(phenylmethoxy)silane, (2-chloroethyl)tris(2-methoxyethoxy) silane, and 1-aminocyclopropane-1-carboxylic acid.

4. The method of claim 3, wherein the ethylene-releasing compound is 2-Chloroethylphosphonic acid.

5. The method of claim 1, wherein the betalains of step c) are harvested from at least 100 beet plants.

6. The method of claim 1, wherein the amount of betalain pigment isolated in in step c) is an amount of betalain pigment at least 15% higher (w/w) as compared to the amount of betalain pigment that is obtained in a control experiment without the use of an ethylene-releasing compound.

7. The method of claim 1, wherein the amount of ethylene-releasing compound applied by spraying in step a) is an amount between about 360 g/h to about 1080 g/h.

8. The method of claim 7, wherein the amount of ethylene-releasing compound is about 720 g/h.

9. The method of claim 1, wherein the foliar spraying of ethylene-releasing compound of step a) is done at 2 weeks after planting.

10. The method of claim 1, wherein the foliar spraying of ethylene-releasing compound of step a) is done later than 2 weeks after planting.

11. The method of claim 1, wherein the foliar spraying of ethylene-releasing compound of step a) is done at least 3 times before the harvesting the beet plants or wherein harvesting of the beet plants is done later than 6 weeks after planting.

12. The method of claim 1, wherein the isolating of the betalains is done by extraction of the betacyanins and betaxanthins pigments from the taproots of the harvested beet plants.

13. The method of claim 1, wherein the obtained betalain pigment color composition comprises at least 20% dry matter (w/w) of the isolated betalain pigments.

14. A betalain pigment color composition obtained by the method of claim 1.

15. The use of a betalain pigment color composition obtained according to the method of claim 1 for coloring of an edible product or a pharmaceutical product.

16. The use of claim 15, wherein the edible product is a food product selected from a dairy product, juice, liquid beverage, powder beverage, confectionery, baked goods, processed foods, wine gum, marmalade, jam, sugar confectionery, panned chocolate lentils, sausage casings, pasta, macaroni, cheese, prepared food or extruded foods, and pet food.
